(19) United States
(12) Patent Application Publication
(10) Pub. No.: US 2010/0331942 A1
(43) Pub. Date: Dec. 30, 2010

(54) MRI COMPATIBLE IMPLANTABLE MEDICAL LEAD AND METHOD OF MAKING SAME

(75) Inventors: Martin Cholette, Acton, CA (US); Scott Salys, Plano, TX (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/493,984

(22) Filed: Jun. 29, 2009

(51) Int. Cl. A61N 1/05; H01R 43/00

(52) U.S. Cl. 607/127; 29/825

(57) ABSTRACT

An implantable medical lead is disclosed herein. The implantable medical lead may include a body including an electrical insulation tube, a distal portion with an electrode, and a proximal portion with a lead connector end. The electrical insulation tube may be coaxial with a longitudinally extending center axis of the body. The lead may also include an electrical pathway extending between the electrode and lead connector end, the electrical pathway including an inductor comprising an electrical conductor helically wound directly on an outer circumferential surface of the insulation tube.

### FIELD OF THE INVENTION

The present invention relates to medical apparatus and methods. More specifically, the present invention relates to implantable medical leads for and methods of manufacturing such leads.

### BACKGROUND OF THE INVENTION

Existing implantable medical leads for use with implantable pulse generators, such as neurostimulators, pacemakers, defibrillators or implantable cardioverter defibrillators (“ICD”), are prone to heating and induced current when placed in the strong magnetic (static, gradient and RF) fields of a magnetic resonance imaging (“MRI”) machine. The heating and induced current are the result of the lead acting like an antenna in the magnetic fields generated during a MRI. Heating and induced current in the lead may result in deterioration of stimulation thresholds or, in the context of a cardiac lead, even increase the risk of cardiac tissue damage and perforation.

Over fifty percent of patients with an implantable pulse generator and implanted lead require, or can benefit from, a MRI in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI are only going to grow over time. For example, MRI has been proposed as a visualization mechanism for lead implantation procedures.

There is a need in the art for an implantable medical lead configured for improved MRI safety. There is also a need in the art for methods of manufacturing and using such a lead.

### BRIEF SUMMARY OF THE INVENTION

An implantable medical lead is disclosed herein. In one embodiment, the implantable medical lead may include a body including an electrical insulation tube, a distal portion with an electrode, and a proximal portion with a lead connector end. The electrical insulation tube may be coaxial with a longitudinally extending center axis of the body. The lead may also include an electrical pathway extending between the electrode and lead connector end, the electrical pathway including an inductor comprising an electrical conductor helically wound directly on an outer circumferential surface of the insulation tube.

In another embodiment, there is disclosed a method of manufacturing an implantable medical lead. In one embodiment, the method may include: providing an inner tube, wherein, when the lead is completed, the inner tube forms a most radially inward insulation layer of the lead; forming a coiled inductor on an outer circumferential surface of the inner tube by helically winding an electrical conductor directly on the outer circumferential surface; electrically connecting at least one of a linearly extending conductor and a helically routed conductor to the inductor; and electrically connecting an electrode to the inductor in an arrangement that causes electricity traveling to the electrode from the at least one of a linearly extending conductor and a helically routed conductor to pass through the inductor.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of an implantable medical lead and a pulse generator for connection thereto.

FIG. 2 is a longitudinal cross-section of a lead distal end.

FIG. 3 is an isometric view of a CRT lead distal end having a coiled inductor wound about an inner tubing, wherein a portion of FIG. 3 is enlarged cut-away view of the coiled conductor forming the inductor, the cut-away view depicting the conductive core and electrical insulation jacket of some versions of the coiled conductor.

FIG. 4 is an isometric view of the CRT lead distal end of FIG. 3 including an electrode coupled to the coiled inductor.

FIG. 5 illustrates the CRT lead distal end of FIG. 4 coated in reflowed insulative material.

FIG. 6 illustrates an inductor sub-assembly of the CRT lead distal end.

FIG. 7 illustrates a multi-polar CRT lead distal end having four inductor sub-assemblies.

### DETAILED DESCRIPTION

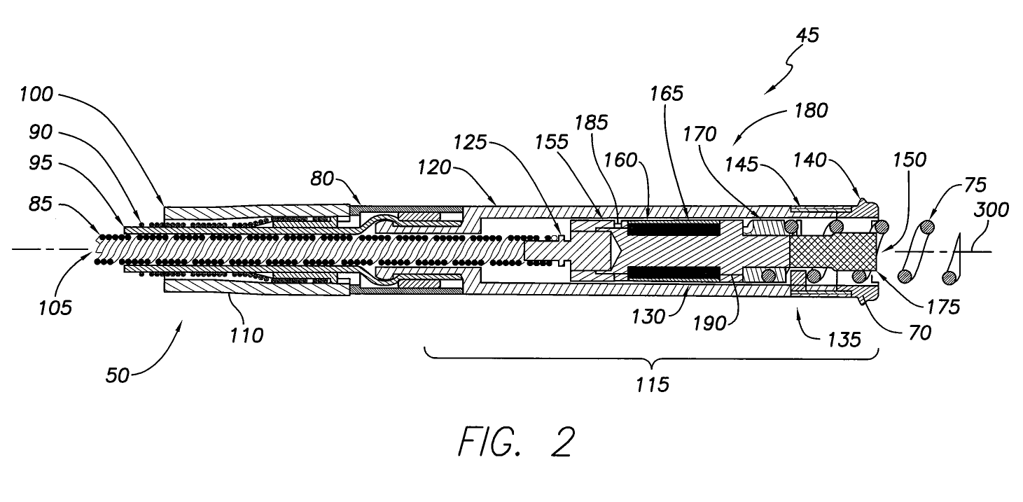

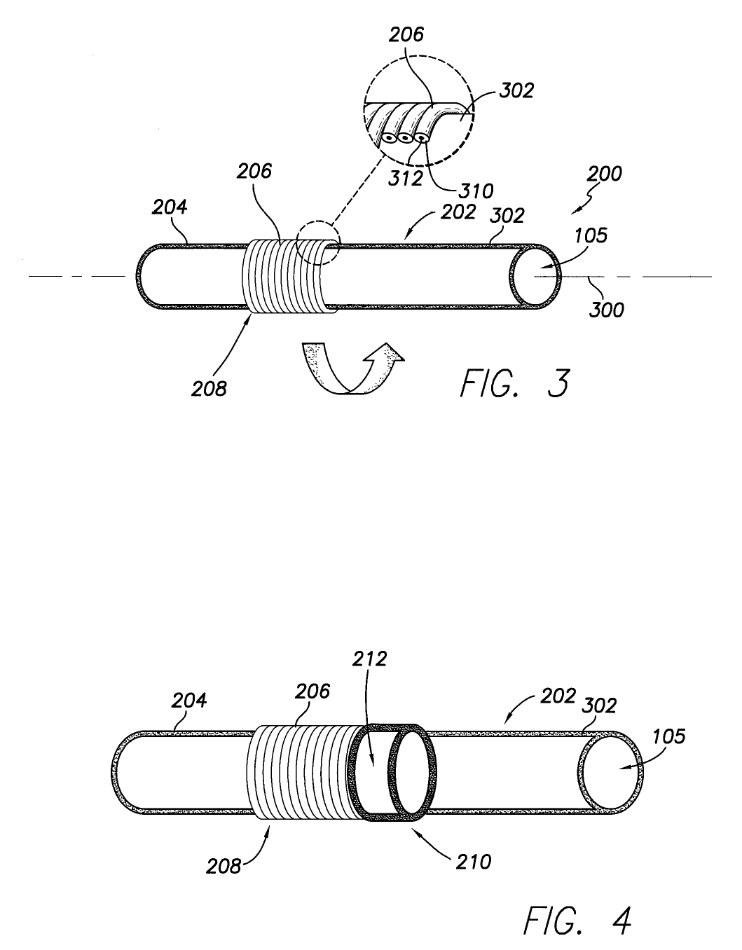

Disclosed herein is an implantable medical lead employing an inductor 208 near the distal end 45 of the lead, wherein the lead is manufactured and configured to have a reduced diameter and improved flexibility as compared to other inductor equipped medical leads. More specifically, the implantable medical lead may even be of an appropriate French size and flexibility that will readily allow its use in cardiac resynchronization therapy (CRT). As will be understood from the discussion given below with respect to FIGS. 3-7, to achieve reduced French sizes and increased flexibility, an inner tubing of the lead is used to helically wind the inductor wire 206, rather than, for example, helically winding about a bobbin, as discussed below with respect to FIG. 2. The inductor 208 may have a self-resonating frequency at approximately 64 MHz and 128 MHz to filter MRI energy.

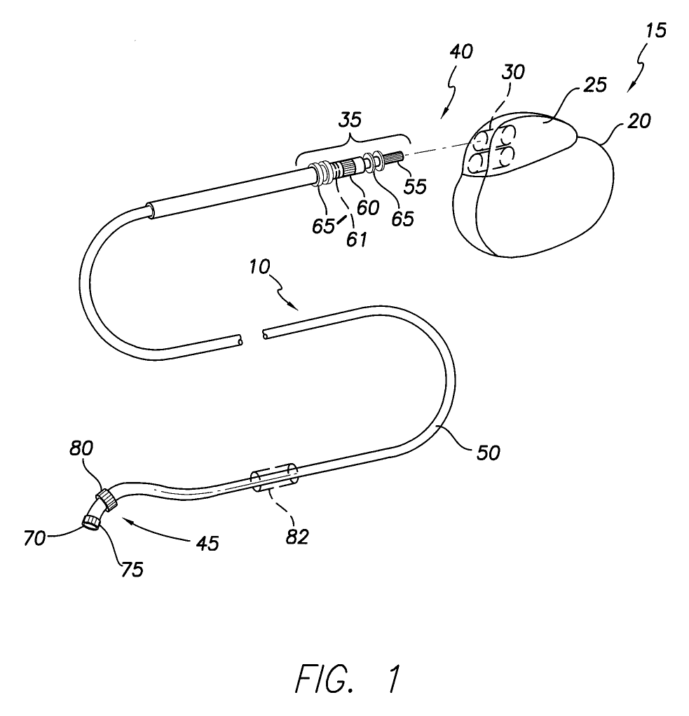

For a general discussion of an embodiment of a lead 10 employing a coil inductor 160, reference is made to FIG. 1, which is an isometric view of the implantable medical lead 10 and a pulse generator 15 for connection thereto. The pulse generator 15 may be a pacemaker, defibrillator, ICD or neurostimulator. As indicated in FIG. 1, the pulse generator 15 may include a can 20, which may house the electrical components of the pulse generator 15, and a header 25. The header may be mounted on the can 20 and may be configured to receive a lead connector end 35 in a lead receiving receptacle 30. Although only a single lead is illustrated, it can be appreciated that multiple leads may be implemented. In particular, for example, for CRT treatments, there may be leads for both the right and left ventricle.

As shown in FIG. 1, in one embodiment, the lead 10 may include a proximal end 40, a distal end 45 and a tubular body 50 extending between the proximal and distal ends. In some embodiments, the lead may be a 6 French lead. In other embodiments, the lead 10 may be of other sizes and models. The lead 10 may be configured for a variety of uses. For example, the lead 10 may be a RA lead, RV lead, LV Brady lead, RV Tachy lead, intrapericardial lead, etc.

As indicated in FIG. 1, the proximal end 40 may include a lead connector end 35 including a pin contact 55, a first ring contact 60, a second ring contact 61, which is optional, and sets of spaced-apart radially projecting seals 65. In some embodiments, the lead connector end 35 may include the same or different seals and may include a greater or lesser number of contacts. The lead connector end 35 may be received in a lead receiving receptacle 30 of the pulse generator 15 such that the seals 65 prevent the ingress of bodily fluids into the respective receptacle 30 and the contacts 55, 60, 61 electrically contact corresponding electrical terminals within the respective receptacle 30.

As illustrated in FIG. 1, in one embodiment, the lead distal end 45 may include a distal tip 70, a tip electrode 75 and a ring electrode 80. In some embodiments, the lead body 50 is configured to facilitate passive fixation and/or the lead distal end 45 includes features that facilitate passive fixation. In such embodiments, the tip electrode 75 may be in the form of a ring or domed cap and may form the distal tip 70 of the lead body 50.

As shown in FIG. 2, which is a longitudinal cross-section of the lead distal end 45, in some embodiments, the tip electrode 75 may be in the form of a helical anchor 75 that is extendable from within the distal tip 70 for active fixation and serving as a tip electrode 75.

As shown in FIG. 1, in some embodiments, the distal end 45 may include a defibrillation coil 82 about the outer circumference of the lead body 50. The defibrillation coil 82 may be located proximal of the ring electrode 70.

The ring electrode 80 may extend about the outer circumference of the lead body 50, proximal of the distal tip 70. In other embodiments, the distal end 45 may include a greater or lesser number of electrodes 75, 80 in different or similar configurations.

As can be understood from FIGS. 1 and 2, in one embodiment, the tip electrode 75 may be in electrical communication with the pin contact 55 via a first electrical conductor 85, and the ring electrode 80 may be in electrical communication with the first ring contact 60 via a second electrical conductor 90. In some embodiments, the defibrillation coil 82 may be in electrical communication with the second ring contact 61 via a third electrical conductor. In yet other embodiments, other lead components (e.g., additional ring electrodes, various types of sensors, etc.) (not shown) mounted on the lead body distal region 45 or other locations on the lead body 50 may be in electrical communication with a third ring contact (not shown) similar to the second ring contact 61 via a fourth electrical conductor (not shown). Depending on the embodiment, any one or more of the conductors 85, 90 may be a multi-strand or multi-filar cable or a single solid wire conductor run singly or grouped, for example in a pair.

As shown in FIG. 2, in one embodiment, the lead body 50 proximal of the ring electrode 80 may have a concentric layer configuration and may be formed at least in part by inner and outer helical coil conductors 85, 90, an inner tubing 95, and an outer tubing 100. The helical coil conductor 85, 90, the inner tubing 95 and the outer tubing 100 form concentric layers of the lead body 50. The inner helical coil conductor 85 forms the inner most layer of the lead body 50 and defines a central lumen 105 for receiving a stylet or guidewire therethrough. The inner helical coil conductor 85 is surrounded by the inner tubing 95 and forms the second most inner layer of the lead body 50. The outer helical coil conductor 90 surrounds the inner tubing 95 and forms the third most inner layer of the lead body 50. The outer tubing 100 surrounds the outer helical coil conductor 90 and forms the outer most layer of the lead body 50.

In one embodiment, the inner tubing 95 may be formed of an electrical insulation material such as, for example, ethylene tetrafluoroethylene (“ETFE”), polytetrafluoroethylene (“PTFE”), silicone rubber, silicone rubber polyurethane copolymer (“SPC”), or etc. The inner tubing 95 may serve to electrically isolate the inner conductor 85 from the outer conductor 90. The outer tubing 100 may be formed of a biocompatible electrical insulation material such as, for example, silicone rubber, silicone rubber-polyurethane-copolymer (“SPC”), polyurethane, gore, or etc. The outer tubing 100 may serve as the jacket 100 of the lead body 50, defining the outer circumferential surface 110 of the lead body 50.

As illustrated in FIG. 2, in one embodiment, the lead body 50 in the vicinity of the ring electrode 80 transitions from the above-described concentric layer configuration to a header assembly 115. For example, in one embodiment, the outer tubing 100 terminates at a proximal edge of the ring electrode 80, the outer conductor 90 mechanically and electrically couples to a proximal end of the ring electrode 80, the inner tubing 95 is sandwiched between the interior of the ring electrode 80 and an exterior of a proximal end portion of a body 120 of the header assembly 115, and the inner conductor 85 extends distally past the ring electrode 80 to electrically and mechanically couple to components of the header assembly 115 as discussed below.

As depicted in FIG. 2, in one embodiment, the header assembly 115 may include the body 120, a coupler 125, an inductor assembly 130, and a helix assembly 135. The header body 120 may be a tube forming the outer circumferential surface of the header assembly 115 and enclosing the components of the assembly 115. The header body 120 may have a soft atraumatic distal tip 140 with a radiopaque marker 145 to facilitate the soft atraumatic distal tip 140 being visualized during fluoroscopy. The distal tip 140 may form the extreme distal end 70 of the lead 10 and includes a distal opening 150 through which the helical tip anchor 75 may be extended or retracted. The header body 120 may be formed of polyetheretherketone (“PEEK”), polyurethane, or etc., the soft distal tip 140 may be formed of silicone rubber, SPC, or etc., and the radiopaque marker 145 may be formed of platinum, platinum-iridium alloy, tungsten, tantalum, or etc.

As indicated in FIG. 2, in one embodiment, the inductor assembly 130 may include a bobbin 155, a coil inductor 160 and a shrink tube 165. The bobbin 155 may include a proximal portion that receives the coupler 125, a barrel portion about which the coil inductor 160 is wound, and a distal portion coupled to the helix assembly 135. The bobbin 155 may be formed of an electrical insulation material such as PEEK, polyurethane, or etc.

As illustrated in FIG. 2, the shrink tube 165 may extend about the coil inductor 160 to generally enclose the coil inductor 160 within the boundaries of the bobbin 155 and the shrink tube 165. The shrink tube 165 may act as a barrier between the coil inductor 160 and the inner circumferential surface of the header body 120. Also, the shrink tube 165 may be used to form at least part of a hermitic seal about the coil inductor 160. The shrink tube 165 may be formed of fluorinated ethylene propylene (“FEP”), polyester, or etc.

As shown in FIG. 2, a distal portion of the coupler 125 may be received in the proximal portion of the bobbin 155 such that the coupler 125 and bobbin 155 are mechanically coupled to each other. A proximal portion of the coupler 125 may be received in the lumen 105 of the inner coil conductor 85 at the extreme distal end of the inner coil conductor 85, the inner coil conductor 85 and the coupler 125 being mechanically and electrically coupled to each other. The coupler 125 may be formed of MP35N, platinum, platinum iridium alloy, stainless steel, or etc.

As indicated in FIG. 2, the helix assembly 135 may include a base 170, the helical anchor electrode 75, and a steroid plug 175. The base 170 forms the proximal portion of the assembly 135. The helical anchor electrode 75 forms the distal portion of the assembly 135. The steroid plug 175 may be located within the volume defined by the helical coils of the helical anchor electrode 75. The base 170 and the helical anchor electrode 75 are mechanically and electrically coupled together. The distal portion of the bobbin 155 may be received in the helix base 170 such that the bobbin 155 and the helix base 170 are mechanically coupled to each other. The base 170 of the helix assembly 135 may be formed of platinum, platinum-iridium alloy, MP35N, stainless steel, or etc. The helical anchor electrode 75 may be formed of platinum, platinum-iridium ally, MP35N, stainless steel, or etc.

As illustrated in FIG. 2, a distal portion of the coupler 125 may be received in the proximal portion of the bobbin 155 such that the coupler 125 and bobbin 155 are mechanically coupled to each other. A proximal portion of the coupler 125 may be received in the lumen 105 of the inner coil conductor 85 at the extreme distal end of the inner coil conductor 85 such that the inner coil conductor 85 and the coupler 125 are both mechanically and electrically coupled to each other. The coupler 125 may be formed of MP35N, stainless steel, or etc.

As can be understood from FIG. 2 and the preceding discussion, the coupler 125, inductor assembly 130, and helix assembly 135 are mechanically coupled together such that these elements 125, 130, 135 of the header assembly 115 do not displace relative to each other. Instead these elements 125, 130,135 of the header assembly 115 are capable of displacing as a unit relative to, and within, the body 120 when a stylet or similar tool is inserted through the lumen 105 to engage the coupler 125. In other words, these elements 125, 130,135 of the header assembly 115 form an electrode-inductor assembly 180, which can be caused to displace relative to, and within, the header assembly body 120 when a stylet engages the proximal end of the coupler 125. Specifically, the stylet is inserted into the lumen 105 to engage the coupler 125, wherein rotation of the electrode-inductor assembly 180 via the stylet in a first direction causes the electrode-inductor assembly 180 to displace distally, and rotation of the electrode-inductor assembly 180 via the stylet in a second direction causes the electrode-inductor assembly 180 to retract into the header assembly body 120. Thus, causing the electrode-inductor assembly 180 to rotate within the body 120 in a first direction causes the helical anchor electrode 75 to emanate from the tip opening 150 for screwing into tissue at the implant site. Conversely, causing the electrode-inductor assembly 180 to rotate within the body 120 in a second direction causes the helical anchor electrode 75 to retract into the tip opening 150 to unscrew the anchor 75 from the tissue at the implant site.

As already mentioned and indicated in FIG. 2, the coil inductor 160 may be wound about the barrel portion of the bobbin 155. A proximal end 185 of the coil inductor 160 may extend through the proximal portion of the bobbin 155 to electrically couple with the coupler 125, and a distal end 190 of the coil inductor 160 may extend through the distal portion of the bobbin 155 to electrically couple to the helix base 170. Thus, in one embodiment, the coil inductor 160 is in electrical communication with the both the inner coil conductor 85, via the coupler 125, and the helical anchor electrode 75, via the helix base 170. Therefore, the coil inductor 160 acts as an electrical pathway through the electrically insulating bobbin 155 between the coupler 125 and the helix base 170. In one embodiment, all electricity destined for the helical anchor electrode 75 from the inner coil conductor 85 passes through the coil inductor 160 such that the inner coil conductor 85 and the electrode 75 both benefit from the presence of the coil inductor 160, the coil inductor 160 acting as a lumped inductor 160 when the lead 10 is present in a magnetic field of a MRI.

As the helix base 170 may be formed of a mass of metal, the helix base 170 may serve as a relatively large heat sink for the inductor coil 160, which is physically connected to the helix base 170. Similarly, as the coupler 125 may be formed of a mass of metal, the coupler 125 may serve as a relatively large heat sink for the inductor coil 160, which is physically connected to the coupler 125.

While the lead 10 of FIG. 2 may be well suited for use in the right atrium or right ventricle, the stiffness provided by the bobbin 155, as well as the relatively large size of the lead 10, attributable in part to the inductor structures, may make it difficult to implement as a left ventricular lead for biventricular pacing. Generally, pacemaker leads, such as lead 10, are passed through the subclavian vein into the right atrium and/or right ventricle. However, in biventricular pacing, an additionally lead may be passed through another vein, the coronary sinus, to reach the left ventricle. Specifically, the left ventricular lead may be passed through a small hole called the “os” of the coronary sinus. In order to do so, the left ventricular lead is manipulated, i.e., bent at a relatively sharp angle, upon exiting the subclavian vein. To facilitate the passing of the lead through the os of the coronary sinus, the left ventricular lead may be smaller and more flexible than the previously described lead 10, while still providing MRI compatibility.

The following discussion describes an implantable medical lead that may achieve appropriate French sizes and provide flexibility with MRI compatibility. To achieve MRI compatibility, a left ventricular CRT lead having one self resonating inductor per electrode may be provided. More particularly, an inner tube of the left ventricular lead may be used as a spindle on which a self resonating inductor may be wound. That is to say, an inner tube 95, such as that depicted in the lead of FIG. 2, may be used as a spindle on which the coils of the inductor are directly wound. For a discussion of such a MRI compatible lead embodiment and a step-wise method of manufacturing such a lead, reference is now made to FIGS. 3-7, which are simplified diagrammatic drawings of inductor sub assemblies 200 wherein an inner tube similar to that depicted in FIG. 2 as tube 95 will now be referred to as inner tube 204. While the lead configuration described below is useful for any type of application, it may be especially useful in the context of a left ventricular lead to be used for CRT.

FIG. 3 illustrates a lead body 202 including an inner tubing 204 similar to the inner tubing 95 of the lead 10 described above. In particular, the inner tubing 204 may be layered concentrically over an inner helical coil conductor (not shown). Hence, the helical coil conductor and inner tubing 202 form concentric layers of the lead body 202. The helical coil conductor (not shown) may form an inner most layer of the lead body 202 and define a central lumen for receiving a stylet or guidewire therethrough. As previously described, the inner tubing 204 may be formed of an electrical insulation material such as, for example, ETFE, PTFE, silicone rubber, SPC, etc. The inner tubing 204 may serve to electrically isolate the inner conductor (not shown).

A wire 206 may be wound around the inner tubing 204 to form a coil inductor 208. Hence, the inner tubing 204 serves as a mandrel on which the inductor 208 can be wound. In one embodiment, the wire 206 may be wound around the inner tubing 204 approximately 50 to 75 times to achieve a desired self-resonant frequency. The wire 206 used to form the inductor 208 may be a high conductivity, biocompatible wire including 20 to 90 percent cored conductive material. In one embodiment, the wire may be 0.0002 of a inch in diameter, i.e., #44 gage silver cored MP35N, commonly referred to as DFT wire. For example, the wire may be approximately 50 to 75 percent silver core DFT wire. Additionally, or alternatively, the wire 206 may be coated with high dielectric strength material, such as PTFE, for example, for electrical insulation.

Several factors may influence the self resonant frequency of the inductor 208. For example, thickness of wire coating, the diameter of the inductor coil, the length of the inductor 208, and the pitch of the inductor coil. The inductor coil may have a diameter between approximately 2 French (0.026″ or 0.67 mm) and approximately 9 French (0.118″ or 3 mm). The length of the inductor may be between approximately 0.25 cm and approximately 3 cm, and the pitch of the inductor may be between approximately 0.0015″ and approximately 0.010″. It will be appreciated by those of skill in the art that there may be additional factors that influence the self-resonant frequency and, further, that the various factors may be taken into account to achieve a desirable frequency response.

As illustrated in FIG. 4, an electrode 210 may be installed over the inner tube 204. The electrode 210 may have a generally annular shape, or other shape, so that it may be inserted over the inner tubing 204 and moved longitudinally over the inner tubing 204 to a location relative to the inductor 208. Once in place, the electrode 210 may be electrically coupled to the inductor 208 via conductor 212. For example, the electrode 210 may be welded or crimp-welded to a distal end of inductor 208 to provide both electrical and mechanical coupling of the electrode 210 and the inductor 208. In one embodiment, the electrode may be made of platinum, platinum-iridium alloy, stainless steel, MP35N, etc., have an inner diameter of between approximately 0.020″ and approximately 0.117″ and an outer diameter of between approximately 0.025″ and approximately 0.12″.

As illustrated in FIG. 4, the electrode 210 may be located immediately proximal to the distal end of the inductor 208. In other embodiments, the electrode 210 may overlap a portion of the distal end of the inductor 208. In yet another alternative embodiment, the electrode 210 may be located some distance along the length of the lead from the distal end of the inductor 208. As previously mentioned, in conjunction with conductive members in the lead, the inductor 208 forms a portion of the electrical path between the electrode 210 and the pulse generator 15.

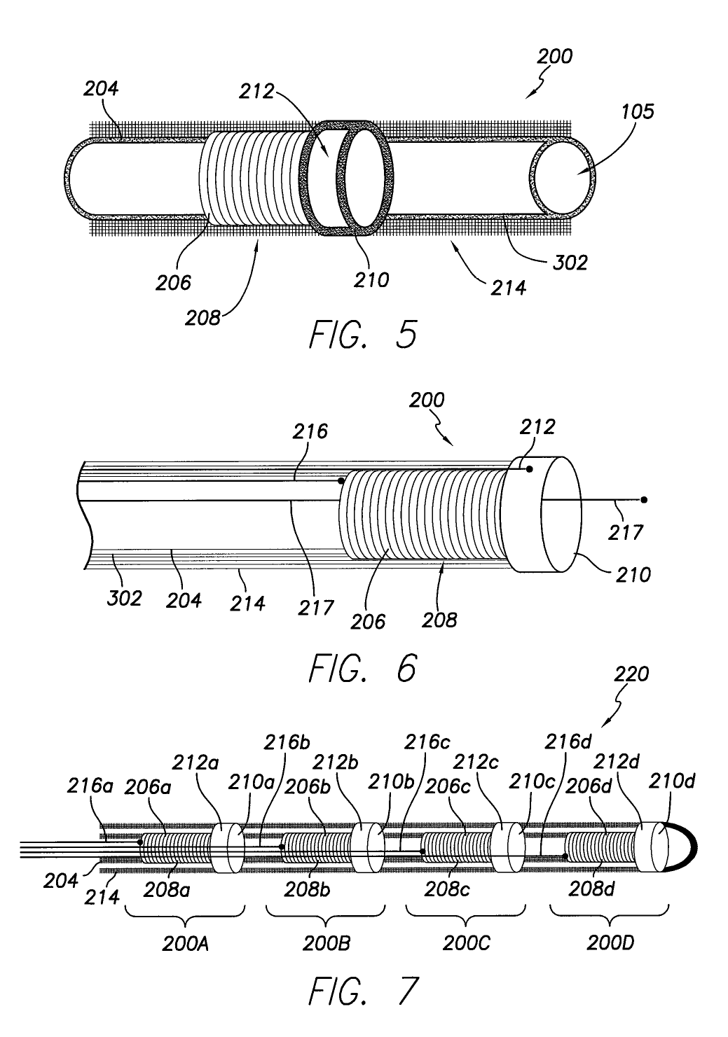

As shown in FIG. 5, after the electrode 210 has been coupled to the inductor 208, the sub-assembly 200 may be covered with reflowed material 214. For example, the sub assembly 200 may be covered with reflowed Optim™ or SPC, silicone rubber or polyurethane. The electrode 210 may still be exposed after the reflowed material 214 is applied. The thickness of the reflowed material 214 may be between approximately 0.004″ and approximately 0.012″. The thickness of the reflowed material 214 may influence the resonant frequency of the inductor 208.

FIG. 6 illustrates the complete sub-assembly 200 having the wire 206 of the inductor 208 coupled to a conductor 216 extending through the lead body from an electrical contact on the lead connector end. Depending on the embodiment, the conductor 216 may be a wire or cable conductor linearly routed through the wall of the lead body or along the inner tube 204. Alternatively, the conductor 216 may be a helically routed conductor similar to either of the conductors 85, 90 depicted in FIG. 2. As depicted in FIG. 6, other conductors 217 may extend through the lead body to other electrodes or devices located distal of the electrode 210, wherein the other conductors 217 are not electrically connected to the conductor 216, inductor 208 or electrode 210.

Multiple sub-assemblies may be provided on a single lead body to create a multipolar lead. For example, a quad polar lead 220 is illustrated in FIG. 7. The quad polar lead 220 may include four sub-assemblies 200A-D assembled on the same inner tube 204. Each sub-assembly 200A-D includes a respective inductor 208a-d formed of a respective wire or wires 206a-d wound about the inner tube 204 and coupled to a respective electrode 21 Oa-d via a respective conductor 212a-d, each respective assembly 200A-D being in electrical communication with a respective electrical contact of the lead connector end 35 via a respective conductor 216a-d extending through the lead body and coupled to a respective inductor 208a-d.

In one embodiment, each sub-assembly 200A-D may be substantially similar. Specifically, the inductor 208a-d of each sub-assembly 200A-D may include approximately the same number of windings, the same pitch and the same length. In other embodiments, one or more of the sub-assemblies 200A-D may be configured differently from one or more of the other sub-assemblies. Specifically, one or more of the inductors 208a-d include at least one of a different number of windings, different pitch and different length. Also, the conductors 216a-d serving the respective inductors 208a-d may be the same type of conductors or different types of conductors (e.g., some conductors 216a-d may be linearly routed wall conductors or helically routed coil conductors.

As can be understood from FIGS. 1, 2 and 6, in one embodiment, the implantable medical lead 10 disclosed herein may include a body 50 including an electrical insulation tube 204, a distal portion 45 with an electrode 210, and a proximal portion 40 with a lead connector end 35. The electrical insulation tube (95 in FIG. 2 and 204 in FIG. 3) may be coaxial with a longitudinally extending center axis 300 of the body 50. As indicated in FIG. 6, the lead may also include an electrical pathway extending between the electrode 210 and lead connector end 35, the electrical pathway including an inductor 208 comprising an electrical conductor 206 helically wound directly on an outer circumferential surface 302 of the insulation tube. In other words, in one embodiment, the electrical conductor 206 forming the inductor may be caused to be helically wound directly onto the outer circumferential surface 302 of the inner tube 204 without anything between the coils of the conductor 206 and the outer circumferential surface 302. Thus, if the conductor 206 does not have its own dedicated insulation jacket (see 310 in enlarged portion of FIG. 3), then the electrically conductive core 312 of the conductor 206 may rest directly on the outer circumferential surface 302. Similarly, if the conductor 206 does have its own dedicated insulation jacket 310, then the insulation jacket 310 of the conductor 206 may rest directly on the outer circumferential surface 302.

As can be understood from FIGS. 1 and 6, the inductor 208 may be electrically coupled to an electrical contact on the lead connector end 35 via a linearly routed conductor 216 in the form of a solid wire or multi-filar cable. In other embodiments, as can be understood from FIGS. 1 and 6, the inductor 208 may be electrically coupled to an electrical contact on the lead connector end 35 via a helically routed coil conductor similar to such coil conductor 85 depicted in FIG. 2. In such an embodiment, while the coil conductor 85 may have a large number of coil turns over its length extending between the distal and proximal ends of the lead body 50, the inductor 208 connected between the coil conductor 85 and electrode 210 may have a substantially fewer number of coil turns, for example, approximately 50 to approximately 75 turns to be tuned to a desired frequency, for example, 64 MHz to 128 MHz.

As can be understood from FIGS. 2-6, the insulation tube (95 in FIG. 2 and 204 in FIG. 3-6) may form a most radially inner insulation layer of the lead body 50. As can be understood from FIG. 3, the insulation tube may define a lumen 105 extending therethrough. As indicated in FIG. 2, the insulation tube 95 my circumferentially extend about a helically coiled electrical conductor 85, and the conductor 85 may define a lumen extending therethrough.

As indicated in FIGS. 5-6, the lead may further include a layer 214 of material reflowed directly over the inductor 208. The reflowed material 214 may include at least one of silicone rubber, polyurethane and SPC.

The electrode 210 may be located near a distal end of the inductor 208 as indicated in FIGS. 3-6. The electrical conductor 206 helically wound to form the inductor 208 may include at least one of 75 percent silver core wire and DFT.

As shown FIG. 3 in the enlarged cut-away view the coiled conductor 206 forming the inductor 208, in some embodiments the conductor 206 may include a conductive core 312 and a dedicated electrical insulation jacket 310. In some embodiments, the inductor 208 includes approximately 50 to approximately 75. turns of the electrical conductor 206 helically wound to form the inductor 206.

Although the present invention has been described with reference to preferred embodiments, persons skilled in the art will recognize that changes may be made in form and detail without departing from the spirit and scope of the invention.

What is claimed is:

1. An implantable medical lead comprising:
a body including an electrical insulation tube, a distal portion with an electrode, and a proximal portion with a lead connector end, wherein the electrical insulation tube is coaxial with a longitudinally extending center axis of the body; and
an electrical pathway extending between the electrode and lead connector end and including an inductor comprising an electrical conductor helically wound directly on an outer circumferential surface of the insulation tube.

2. The lead of claim 1, wherein the insulation tube forms a most radially inner insulation layer of the body.

3. The lead of claim 1, wherein the insulation tube defines a lumen extending therethrough.

4. The lead of claim 1, further comprising a helically coiled electrical conductor about which the insulation tube circumferentially extends.

5. The lead of claim 4, wherein the helically coiled electrical conductor defines a lumen extending therethrough.

6. The lead of claim 1, further comprising a layer of material reflowed directly over the inductor.

7. The lead of claim 6, wherein the reflowed material includes at least one of silicone rubber, polyurethane and SPC.

8. The lead of claim 1, wherein the electrode is located near a distal end of the inductor.

9. The lead of claim 1, wherein the electrical conductor helically wound to form the inductor includes at least one of 75 percent silver core wire and DFT.

10. The lead of claim 1, wherein the electrical conductor helically wound to form the inductor includes a dedicated electrical insulation jacket.

11. The lead of claim 1, wherein the inductor comprises approximately 50 to approximately 75 turns of the electrical conductor helically wound to form the inductor.

12. The lead of claim 1, wherein the inductor is configured to have a self resonant frequency of approximately 64 MHz or 128 MHz.

11. The lead of claim 1, further comprising another electrical pathway extending between another electrode and the lead connector end and including another inductor comprising another electrical conductor helically wound directly on the outer circumferential surface of the insulation tube.

12. The lead of claim 11, wherein the inductor and the another inductor have at least one of the same pitch and number of coils.

13. The lead of claim 11, wherein the inductor and the another inductor have different pitches and different numbers of coils.

14. The lead of claim 1, wherein the electrical insulation tube includes at least one of ethylene tetrafluoroethylene (“ETFE”), polytetrafluoroethylene (“PTFE”), silicone rubber, and silicone rubber-polyurethane-copolymer (“SPC”).

16. The lead of claim 1, wherein the electrode is welded or crimp welded to the inductor.

17. The lead of claim 17, wherein the lead is a left ventricular lead for CRT.

18. A method of manufacturing an implantable medical lead, the method comprising:
providing an inner tube, wherein, when the lead is completed, the inner tube forms a most radially inward insulation layer of the lead;
forming a coiled inductor on an outer circumferential surface of the inner tube by helically winding an electrical conductor directly on the outer circumferential surface;
electrically connecting at least one of a linearly extending conductor and a helically routed conductor to the inductor; and
electrically connecting an electrode to the inductor in an arrangement that causes electricity traveling to the electrode from the at least one of a linearly extending conductor and a helically routed conductor to pass through the inductor.

19. The method of claim 18, further comprising reflowing an electrical insulation layer directly over the inductor.

20. The method of claim 19, wherein the reflowed electrical insulation layer includes at least one of silicone rubber, polyurethane and SPC.

21. The method of claim 18, wherein the inner tube includes at least one of ethylene tetrafluoroethylene (“ETFE”), polytetrafluoroethylene (“PTFE”), silicone rubber, and silicone rubber-polyurethane-copolymer (“SPC”).

22. The method of claim 18, wherein the coiled inductor includes between approximately 50 and approximately 75 coil turns.

23. The method of claim 18, wherein the coiled inductor is coiled to be tuned to achieve a self resonant frequency of approximately 64 MHz or approximately 128 MHz.
